(19) United States
(12) Patent Application Publication
(10) Pub. No.: US 2013/0004972 A1
(43) Pub. Date: Jan. 3, 2013

(54) METHOD FOR THE DETECTION OF PREDIABETES

(76) Inventor: Toshiaki Watanabe, Fukuoka-shi (JP)

(21) Appl. No.: 13/519,458

(22) PCT Filed: Dec. 24, 2010
(86) PCT No.: PCT/JP2010/073453

§ 371 Date: Jun. 27, 2012

(30) Foreign Application Priority Data
Dec. 28, 2009 (JP) 2009-297509
Nov. 2, 2010 (JP) 2010-246867

(51) Int. Cl. G01N 33/566; G01N 33/577

(52) U.S. Cl. 435/7.92; 436/501

(57) ABSTRACT

Provided is a method for prediabetes screening by means of methylglyoxal-modified arginine derivative assay, with which it is possible to treat many samples as simply and as safely as with blood sugar assay, and to collect a blood sample taken during a primary health screening in one procedure without imposing any time restraints, complications or risks on the subject. The method for prediabetes screening by means of methylglyoxal-modified arginine derivative assay comprises assaying the methylglyoxal-modified arginine derivative in blood using an assay system which employs an antibody that specifically recognizes methylglyoxal-modified arginine derivative.

### TECHNICAL FIELD

The present invention relates to a method for the detection of prediabetes. More specifically, the present invention relates to a method for the detection of prediabetes, which permits a compact detection of insulin resistance or impaired glucose tolerance particularly in prediabetes by measuring a glycosylated protein including, for example, organic substances such as argpyrimidine compounds possessing a pyrimidine structure or hydroimidazolone compounds possessing a hydroimidazolone structure, as a blood marker, as well as a method for the detection of prediabetes on the basis of the detection of the insulin resistance or impaired glucose tolerance at the stage when a primary medical examination is to be performed.

### BACKGROUND TECHNOLOGY

The worldwide number of patients suffering from diabetes is estimated to outnumber four hundred millions up to the year 2030 from the current number of two hundred eighty-five millions. The ninety percent of the total number of the diabetic patients accounts for patients with obese diabetes caused mainly by lifestyle diseases. In this country, the number of patients with obese diabetes is currently estimated to exceed approximately ten millions, and it is increasing terribly from year to year.

The number of the diabetic patients, however, who undergo a treatment of diabetes, is less than three millions, and patients who develop nephropathy are increasing by ten thousands every year due to a delayed treatment as a result of a failure to receive a sufficient examination and a long-term neglect.

In addition, recent year, among patients with prediabetes (so-called “reserved diabetic patients”), it has been pointed out that the number of those who cause cardiovascular disturbances by an occurrence of an extremely high postprandial blood glucose level (so-called “hidden diabetes”) due to impaired glucose tolerance (IGT), is increasing, even if the fasting plasma glucose would be at a normal level. The American Diabetes Association and The World Health Organization (WHO) have defined such prediabetes as an authentic disease and arouse a significance of improvements of the IGT for the prediabetes by qualitative improvements of lifestyle and by medical treatment. In Japan, it is easy to imagine that the number of patients with diabetes increases rapidly and terribly in the very near future because patients suffering from prediabetes are currently estimated to outnumber ten millions.

The Japan Diabetes Society directs measures for the measurement for a fasting plasma glucose (FPG) level by the primary health examination and for the oral glucose tolerance (OGT) by the secondary health examination as a standard for the diagnosis of prediabetes and diabetes. As it is difficult to accurately diagnose the IGT of prediabetic patients solely by the FPG test or the glycohemoglobin (HbA1c) test of the primary health examination, those who are suspected to suffer from prediabetes by the primary health examination are recommended to undergo an oral glucose tolerance test (OGTT) by the secondary health examination. It is true to state that the OGTT by the secondary health examination may impose a great burden particularly on persons of middle and old age who would be pressed with work because they are required to take a day off for an examination and they are administered with glucose causing risks to temporarily cause a high blood glucose level. It has to be noted herein, however, that potential prediabetic patients who would have neglected the secondary health examination may be advanced towards an authentic diabetes without being conscious of diabetes or its complications by themselves and undergoing any treatment, eventually developing severe complications or leading to the renal dialysis.

With the fact taken into consideration that, although the current cost of medical treatment solely for the renal dialysis for patients with diabetes has already reached several trillion yen, approximately half the number of such diabetic patients dies of ischemic diseases such as myocardial infarction or cerebral infarction originating from diabetes as a primary disease. Therefore, it is the great concern that the cost of medical treatment for diabetes and its complications would undoubtedly reach a tremendously high level unless any effective measures would otherwise be taken.

As described above, the primary health examination is carried out usually by measuring the FPG value, casual blood glucose level and/or HbA1c as an item or items used as criteria for judging diabetes. When the FPG value is 100 mg/dl or higher or the HbA1c value is 5.1% or higher, it is then diagnosed as a suspected insulin resistance or IGT derived therefrom. It is to be noted, however, that, as a high postprandial blood glucose value may occur as a major initial symptom for the IGT caused by insulin resistance, any index used for the conventional primary health examination which has been used so far can be said to be insufficient in the term of accuracy.

Therefore, practically, those who are diagnosed to be suspicious of insulin resistance or IGT based on those indexes for decision by the primary health examination are recommended to undergo a secondary health examination including a 75 g oral glucose tolerance test (75 g OGTT), a homeostasis model assessment ratio (HOMA-R) test, etc. to judge the insulin resistance or the IGT derived therefrom. In the event where it is judged by these laborious tests that the insulin resistance or IGT is positive, this case is then decided as prediabetes.

If a blood sample used for judging a blood glucose level by the primary health examination could be used for a test that can judge the prediabetes at substantially the same accurate level as the OGTT, a rate of the detection of patients with prediabetes could elevate to a tremendously high level, leading eventually to no requirements for the OGTT by the secondary health examination. This would enable to strongly encourage the strategy advocated by The World Health Organization (WHO) and others to suppress diabetes by an early examination or treatment of diabetes.

A brief description will be made hereinafter regarding a detection method used for the diagnosis of diabetes by the secondary health examination or thereafter.

### (1) 75 g Oral Glucose Tolerance Test (75 g OGTT):

The 75 g oral glucose tolerance test (75 g OGTT) is performed for the detection of the IGT level at the time of examination by measuring a blood glucose level, a sugar value in urine, a blood insulin value or the like, and observing its periodical change after orally administering a solution containing 75 grams of glucose. The domestic standard of the diagnosis for this OGTT adopts a 2-hour blood glucose level in 2 hours after administration of glucose. This 75 g OGTT has to be performed with great care because this test may take a long time and cause a severely high blood glucose level after the administration of glucose.

### (2) Blood Insulin Value:

Recently, the blood insulin value attracts attention because it may be associated with a metabolic syndrome, although it is not contained as a standard item for the diagnosis of diabetes. A decrease in susceptibility of this blood insulin value, that is, insulin resistance, is considered as a major factor for the IGT of obese diabetes or prediabetes so that patients with prediabetes or obese diabetes, who demonstrate IGT, may cause a blood insulin value higher than healthy persons may. For instance, when a blood insulin concentration is 15 □U/ml or higher at the time of fasting in early morning, the insulin resistance in this case is apparently positive, and the IGT is caused most likely.

### (3) Homeostasis Model Assessment Ratio (HOMA-R):

HOMA-R is considered to be well associated with IGT or the like when the blood glucose level at the time of fasting is 140 mg/dl or less, and it is computed by a relationship of the blood glucose level at the time of fasting with the blood insulin level at the time of fasting as will be described in the following formula:

HOMA-R=Fasting blood insulin value(□U/ml)×Fasting blood glucose level(mg/dl)/405

In the above formula, when HOMA-R is 2.5 or higher, it is judged that the insulin resistance is positive, while when HOMA-R is 1.6 or less, it is judged as normal. It has to be noted that the HOMA-R is not a test item of the primary health examination and it cannot be applied to patients under treatment by insulin.

### (3) Glucose Clamp Method:

The glucose clamp method is a test for the examination of a degree of a decrease of the blood glucose for those who are administered with insulin, that is, a degree of the effect of insulin administered on the blood glucose (the insulin susceptibility), by determining the point at which a stationary value of the blood glucose is sustained by injection of glucose and insulin. This glucose clamp method can be said to be most accurate in the measurement for insulin resistance among the tests which have been used so far; however, the handling procedures are very laborious so that it does not become so popular in general hospitals.

It can be noted herein that the Maillard reaction which is a reaction for forming advanced glycation endproducts (AGEs) known as oxidative stress inducing factors advances in the living body and is associated with a development of aging and complications of diabetes already developed (for example, see Non-patent Literature Document No. 1).

The Maillard reaction is a two-stage reaction consisting of an early reaction stage and a later reaction stage. The reaction of the early stage involves a reaction of a side-chain amino group or an N-terminal amino group of a protein with a carbonyl group of a sugar forming a Shiff base, followed by the formation of Amadori rearrangement compounds. These compounds formed in the living body by the reaction of the early stage may include, for example, HbA1c, glycosylated albumin, and the like, and they are known as involved in various disease conditions, particularly diabetes. On the other hand, the reaction of the later stage involves a reaction for the formation of glycosylated proteins, i.e., so-called advanced glycation endproducts (AGEs), which are the products formed by the reaction of the later stage having at least one of the characteristics including, e.g., fluorescence, browning, intermolecular or intramolecular linkage and biological recognition, through complicated reactions involved in the oxidation, dehydration, condensation, cyclization and so on of the products formed by the reaction of the early stage. In other words, it is known that the amino group of an amino acid, a peptide or a protein can be subjected to a non-enzymatic condensation and glycosylation with an aldehyde group of a reducing sugar, thus yielding a glycosylated amino acid, a glycosylated peptide or a glycosylated protein (collectively referred to as “glycosylated proteins”, too), respectively.

It is also known that methylglyoxal (hereinafter referred to as “MGO”, too), which is one of the reactive carbonyl products obtained by the Maillard reaction of the early stage, is present in a relatively large amount particularly in blood. It is further reported that its serum level is high for patients with diabetes and it is present in a large amount in the ocular lens of rats with diabetes induced by streptozotocin (Non-patent Literature Document Nos. 2 and 3). Moreover, it is reported that MGO forms a fluorescent product by a reaction of the MGO at a level of its in vivo concentration with a protein and the fluorescent product may function as a direct mediator for the formation of AGEs, as well as it may be associated with diabetes and aging (Non-patent Literature Document Nos. 4, 5 and 6) or, further, with insulin resistance and vascular disorders (Non-patent Literature Document Nos. 7 and 8).

The methylglyoxal (MGO) having the above function may cause a tissue injury by inhibiting the enzymatic reaction of a protease or collagenase involved in a tissue reconstruction due to a chemical reaction with a side chain of an amino acid structuring a protein, in particular a basic lysine or arginine side chain, on the one hand, and it is considered to be associated with metabolic toxicity by modifying and inactivating the side chain of the amino acid structuring an active center of a functional protein involved in metabolism and regulation. The MGO may be detoxified by d-lactic acid with the aid of glyoxalase in a normal state. It is considered, however, that, in such a blood glucose-controlled state causing a rise in oxidative stress, the detoxifying mechanism of the MGO may be hindered resulting in a damage to the nerve cells or blood vessels and complications of diabetes such as neurological disorders, retinopathy, nephritis, or the like (for example, see Patent Document No. 1).

Though the measurement for the MGO as a parameter for the detection of complications of diabetes is very useful in the manner as described above, it is extremely difficult to directly measure the MGO because it is very reactive chemically and a variation in its contents in the living body cannot be controlled.

However, a part of the MGO remaining in the living body in an excessive amount without being detoxified and metabolized is reacted with the arginine side chain of a protein due to its high reactivity, resulting in the formation of advanced glycation endproducts (AGEs) including, for example, argpyrimidine compounds (AP) or hydroimidazolone compounds, such as methylglyoxal-modified arginine adducts (for example, see Non-patent Literature Document Nos. 9 and 10). Therefore, an attempt has been made to indirectly measure the MGO by measuring the methylglyoxal-modified arginine adducts.

It is reported that an immunohistological study using a polyclonal antibody to an amino acid modified by the MGO reveals the presence of a site in a human arteriosclerotic lesion which can be stained strongly with this polyclonal antibody (for example, see Non-patent Literature Document No. 11). There are further reports on the results of the measurement for the methylglyoxal-modified arginine adducts using a monoclonal antibody capable of specifically recognizing argpyrimidine (for example, see Patent Document Nos. 1, 3 and 4) to the effect that the methylglyoxal-modified arginine adduct is useful for a specific immunostaining of a portion of the cerebral artery damaged by the diabetic nephropathy or ischemic cerebral infarction (for example, see Non-patent Literature Document No. 12), it may be associated with a site of the onset of diabetic retinopathy (for example, see Non-patent Literature Document No. 13), and it is useful for the detection of the onset of the vascular complications of diabetic patients (for example, see Non-patent Literature Document No. 14).

From the background as described above, it can be considered as very useful from a clinical or analytical point of view to detect glycosylated proteins by specifically detecting argpyrimidines or their sites. Therefore, a method for the measurement of methylglyoxal-modified arginine adduct using the above monoclonal antibody has been presented (for example, see Patent Document Nos. 1, 3 and 5). These patent documents disclose each the possibility of using the methylglyoxal-modified arginine adduct as a marker for measuring the diabetes or diabetic complications which has or have already been developed, i.e., in which blood glucose levels have already elevated obviously. They do not imply nor disclose whatsoever, however, the usefulness of the methylglyoxal-modified arginine adduct as a marker for the diagnosis of prediabetes that may cause the IGT by the insulin resistance.

Immunological studies using antibodies to such AGE derivatives further reveal that the AGE derivatives are positive due to aging, diabetes, diabetic nephropathy, or the like (for example, see Non-patent Literature Document Nos. 15, 16 and 17). It is also reported that one of the AGE derivatives, i.e., carboxymethyllysine, can be used as a marker for the diagnosis of diabetic complications which have already been developed and in which the blood glucose level at the time of fasting or casual blood glucose level has already elevated apparently. There is neither a disclosure nor a hint at all in any prior art, however, about the possibility of using this AGE derivative as a marker for the diagnosis of the prediabetes.

### PRIOR ART DOCUMENTS

### Patent Documents

[Patent Document No. 1] JP 2004-309147 A (JP 4146264 B)
[Patent Document No. 2] JP 9-178740 A
[Patent Document No. 3] JP 11-246600 A (JP. 4013312 B)
[Patent Document No. 4] JP 11-246599 A
[Patent Document No. 5] JP 2004-309147 A (JP 4146264 B)

### Non-patent Literature Documents

[Non-patent Literature Document No. 1] Bio Industry, Vol. 3, No. 7, p. 14, 1996
[Non-patent Literature Document No. 2] Biochem. Pharmacol., Vol. 46, pp. 805-811, 1993
[Non-patent Literature Document No. 3] Clin. Sci., Vol. 87, pp-21-29, 1994
[Non-patent Literature Document No. 4] Biochim. Biophys. Acta., Vol. 1270, pp. 36-43, 1995
[Non-patent Literature Document No. 5] J. Biol. Chem., Vol. 269, pp. 32299-32305, 1994
[Non-patent Literature Document No. 6] J. Biol. Chem., Vol. 267, pp. 4364-4369, 1992
[Non-patent Literature Document No. 7] Diabetes, Vol. 55, pp. 1289-1299, 2006
[Non-patent Literature Document No. 8] J. Cell Biochem., Vol. 103, pp. 1144-1157, 2008
[Non-patent Literature Document No. 9] Archives Of Biochemistry And Biophysics, 1997, Vol. 344, No. 1, pp. 29-36
[Non-patent Literature Document No. 10] Invest 6. Ophthalmol. Vis. Sci., December 2003, vol. 44, no. 12, pp. 5287-5292
[Non-patent Literature Document No. 11] FEBS Letters, Vol. 410, pp. 313-318, 1997
[Non-patent Literature Document No. 12] The Journal Of Biological Chemistry 1999, Vol. 274, No. 26, pp. 18492-18502
[Non-patent Literature Document No. 13] Current Eye Research 2001, Vol. 23, No. 2, pp. 106-115
[Non-patent Literature Document No. 14] The Journal Of Biological Chemistry 1998, Vol. 273, No. 12, pp. 6928-6936
[Non-patent Literature Document No. 15] J. Clin. Invest., 85, 380-384, 1990
[Non-patent Literature Document No. 16] J. Biol. Chem., 263, 3758-3764, 1989
[Non-patent Literature Document No. 17] J. Clin. Invest., 89, 1102-1112, 1992

### SUMMARY OF THE INVENTION

As a result of extensive review and studies in order to develop a technique for effectively, rapidly and precisely performing a diagnosis of prediabetes, the present inventors have found that a measurement for a substance present in a sample of a living body including, but being not limited to, a methylglyoxal-modified arginine derivative such as an argpyrimidine compound (hereinafter referred to as “AP compound”, too) or a hydroimidazolone compound as a diagnostic marker, thereby capable of detecting the insulin resistance or the IGT in an effective, rapid and precise way. Furthermore, they have found a method for the detection of prediabetes, particularly a method for the detection of prediabetes, which can be performed by the primary health examination on the basis of the above method for the detection of the insulin resistance or the IGT thereby.

More specifically, by focusing their review and studies on an investigation of the possibility of an action of the methylglyoxal-modified arginine derivatives such as a methylglyoxal (MGO)-protein conjugate, e.g., an AP compound, as an IGT-inducing factor, the present inventors have found that there is a close relationship between an increase in the methylglyoxal-modified arginine derivative and a development of the IGT, thereby enabling a detection of the IGT by the measurement of the methylglyoxal-modified arginine derivative in the in vivo sample to be applied to the detection of the prediabetes by the primary health examination.

The present inventors have also found that, as a result of an investigation of an association of an increase in the AP compound in the blood with an increase in the visceral fat caused by obesity by using a monoclonal antibody capable of selectively recognizing the methylglyoxal-modified arginine derivative, there is a correlation between an increase in the methylglyoxal-modified arginine derivative present in the blood and an increase in the visceral fat. Therefore, the present invention has been completed on the basis of these findings.

The term “prediabetes” or “prediabetic” as used in this description is intended to mean a pathological symptom in which a development of prediabetes is suspected in the case where the fasting blood glucose level is as normal as in the scope ranging from 100 mg/dl to 110 mg/dl but the blood glucose level after 2 hours of administration of glucose by the oral glucose tolerance test (OGTT) is in the range of 140 to 199 mg/dl, or where the fasting blood glucose level ranges from over 110 mg/dl to not more than 126 mg/dl (New Classification 2010/2009 of the Guidelines of The Japan Diabetes Society). In other words, in Japan, patients with prediabetes are so-called reserved diabetic patients, although The American Diabetes Association and The World Health Organization recognize the prediabetes as an authentic disease having a high risk for currently developing arteriosclerosis by high postprandial blood glucose. In this connection, if the fasting blood glucose level (FBG) is 126 mg/dl or higher, or if the blood glucose level is 200 mg/dl or higher when measured by the OGTT, the pathological condition is diagnosed as diabetes.

Therefore, the object of the present invention is to provide a method for the detection of prediabetes comprising detecting the absence or presence of insulin resistance or IGT particularly in prediabetes by measuring a methylglyoxal-modified arginine derivative such as an AP compound or a hydroimidazolone compound in a biological sample such as a blood sample.

The present invention as its preferred embodiment has the object to provide a method for the detection of prediabetes by measuring the methylglyoxal-modified arginine derivative in the biological sample such as a blood sample by using an assay system using an antibody capable of specifically recognizing the methylglyoxal-modified arginine derivative such as the AP compound or the hydroimidazolone compound, including an assay system using a monoclonal antibody or a polyclonal antibody thereto, preferably an enzyme-linked immunosorbent assay (ELISA) system.

The present invention as its more preferred embodiment has the object to provide a method for the measurement of prediabetes, which comprises a primary antibody reaction step comprising a reaction of the methylglyoxal-modified arginine derivative such as the AP compound or the hydroimidazolone compound in the biological sample with a primary antibody such as an anti-methylglyoxal monoclonal antibody or the like; an immobilization step comprising an immobilization of a protein-methylglyoxal (MGO) conjugate obtained by a reaction of a protein such as blood serum albumin (BSA) with methylglyoxal (MG); a MGO-primary antibody reaction step comprising a reaction of the MGO of the resulting protein-MGO conjugate with the primary antibody in the sample by adding the sample treated in the above primary antibody reaction step to the immobilized protein-MGO conjugate; and a measurement step comprising a measurement of a labeled secondary antibody by a reaction of the primary antibody reacted in the MGO-primary antibody reaction step with the labeled secondary antibody.

The present invention as its more preferred embodiment has the object to provide a method for the detection of prediabetes, which comprises a qualification of a methylglyoxal (MGO) value in the biological sample on the basis of a calibration curve of the methylglyoxal (MGO) value in a standard sample.

The present invention as another mode has the object to provide a method for the detection of prediabetes comprising a detection of prediabetes on the basis of a result of an insulin resistance test or an IGT test for prediabetes. The method for the detection of prediabetes according to the present invention is useful for a test of a blood sample particularly having a standard blood glucose level lower than 110 mg/dl.

The present invention as in another mode has the object to provide an assay kit for detecting prediabetes by the method as described above.

Although the present invention is described herein by using the terms “AP compound(s)” or “argpyrimidine compound(s)” or its related terms as an example, it should be interpreted that they are understood to encompass the hydroimidazolone compounds and other derivatives, unless otherwise set forth herein.

In order to achieve the objects as described above, the present invention provides a method for the detection of prediabetes comprising the measurement of a methylglyoxal-modified arginine derivative as represented by genera formula [I]:

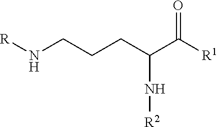

(wherein R is a nitrogen-containing heterocyclic group;
R¹ is a hydrogen atom, a hydroxyl group, a protein residue, or a peptide residue;
R² is a hydrogen atom, an acetyl group, a protein residue, or a peptide residue)
   in a biological sample such as a blood sample.

The present invention as its preferred embodiment provides the method for the detection of prediabetes by the measurement of the methylglyoxal-modified arginine derivative, in which the methylglyoxal-modified arginine derivative [I] is:

an argpyrimidine compound as represented by general formula [II]:

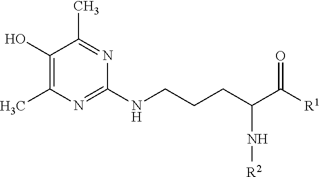

(wherein R¹ and R² have each the same meaning as above); or

a hydroimidazolone compound as represented by general formula [III]:

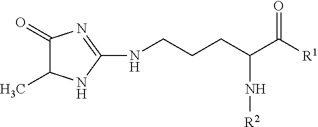

(wherein R¹ and R² have each the same meaning as above); or

a hydroimidazolone compound as represented by general formula [IV]:

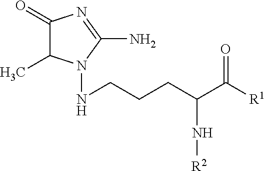

(wherein R¹ and R² have each the same meaning as above); or

a hydroimidazolone compound as represented by general formula [V]:

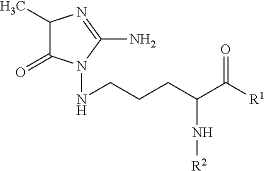

(wherein R¹ and R² have each the same meaning as above).

The present invention as its more preferred embodiment provides the method for the detection of prediabetes in which the argpyrimidine compound [II] is argpyrimidine as represented by structural formula [VI]:

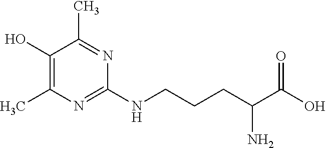 [SMILES: Cc1nc(NCCCC(N)C(=O)O)nc(C)c1O]

or the hydroimidazolone compound [III] is hydroimidazolone as represented by structural formula [VII]:

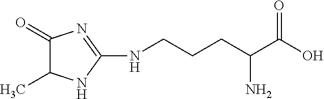 [SMILES: CC1NC(NCCCC(N)C(=O)O)=NC1=O]

The present invention provides the method for the detection of prediabetes comprising the measurement for the methylglyoxal-modified arginine derivative in an in vivo sample such as a blood sample by using an assay system using an antibody capable of specifically recognizing the methylglyoxal-modified arginine derivative such as the AP compound or the hydroimidazolone compound, preferably an assay system using a monoclonal antibody or a polyclonal antibody, or, more preferably an ELISA system.

The present invention as its more specific embodiment provides the method for the measurement of prediabetes, which comprises a primary antibody reaction step comprising a reaction of the argpyrimidine compound in a biological sample with a primary antibody such as an anti-methylglyoxal monoclonal antibody or the like; an immobilization step comprising an immobilization of a blood serum albumin (BSA)-methylglyoxal (MGO) conjugate obtained by a reaction of a blood serum albumin (BSA) with methylglyoxal (MG); a MGO-primary antibody reaction step comprising a reaction of the MGO of the resulting protein-MGO conjugate with the primary antibody in the sample by adding the sample treated in the above primary antibody reaction step to the immobilized BSA-MGO conjugate; and a measurement step comprising the measurement of a labeled secondary antibody by a reaction of the primary antibody reacted in the MGO-primary antibody reaction step with the labeled secondary antibody.

The present invention as its more preferred embodiment provides the method for the detection of prediabetes, which comprises the qualification of the methylglyoxal (MGO) value in the biological sample on the basis of a calibration curve of the methylglyoxal (MGO) value obtained in a standard sample.

The present invention as another mode provides a method for the detection of prediabetes comprising the detection of the presence or absence of prediabetes on the basis of a result of an insulin resistance test or an IGT test for prediabetes. The method for the detection of prediabetes according to the present invention is useful for a test of a blood sample particularly having a standard blood glucose level lower than 110 mg/dl.

The present invention as another mode provides an assay kit for detecting prediabetes by using the method as described above, which comprises components as follows:

Methylglyoxal-modified arginine derivative such as an AP compound or a hydroimidazolone compound; a primary antibody; an immobilized plate with a BSA-MGO conjugate obtained by conjugation between methylglyoxal (MGO) and a bovine serum albumin (BSA); a secondary antibody; as well as a standard antibody (for example, HRP-labeled antibody, etc.) and a standard curve of a standard sample.

The method for the measurement of AP according to the present invention enables the detection of prediabetes in a precise fashion by a single treatment of a blood sample to be used for the primary health examination and further by a simultaneous treatment of multiple samples. The present invention further requires only one single blood collection as have been done by the primary health examination. Moreover, the present invention can solve various issues faced so far with the secondary health examination, resulting in obstacles for the secondary health examination coming into wide use, which include, for example, long binding hours in hospital, laborious procedures, and risks to be caused by loading glucose. As the present invention uses the technique of measuring the AP compound using ELISA or the like for the diagnosis of prediabetes, the diagnosis of prediabetes can be effectively performed in a ready and safe way. In addition, the present invention enables the early diagnosis of potential prediabetes by measuring the insulin resistance or IGT for prediabetes in a precise manner, which is difficult to be detected solely by measuring blood glucose. Therefore, the present invention allows an early treatment of prediabetes in order to make a treatment for inhibiting an advancement of prediabetes and prevent the prediabetes from being developed into diabetes, as advocated by The American Diabetes Association and The World Health Organization. In other words, the present invention can prevent the prediabetes from developing into diabetes or complications, thereby saving a tremendous amount of expenses required for the treatment of diabetes or related diseases and achieving immeasurable effects.

### BRIEF DESCRIPTION OF THE ACCOMPANYING DRAWINGS

FIG. 1 is a graph showing a variation in blood glucose levels of a 10-27-week-old B6 mouse based on a 6-week-old mouse.

FIG. 2 is a graph showing a variation in blood insulin values of a 10-27-week-old B6 mouse based on a 6-week-old mouse.

FIG. 3 is a graph showing a variation in visceral (intra-abdominal) fat amounts of a 10-27-week-old B6 mouse based on a 6-week-old mouse.

FIG. 4 is a graph showing a variation in blood AP values of a 10-27-week-old B6 mouse based on a 6-week-old mouse.

FIG. 5 is a graph showing a periodical change of blood glucose level before (minute 0) and after glucose tolerance of a control rat group and a fructose-dosed rat group.

FIG. 6 is a graph showing blood AP values of a control rat group and a fructose-dosed rat group.

### MODES FOR CARRYING OUT THE INVENTION

The present invention provides a method for the detection of prediabetes by measuring a methylglyoxal-modified arginine derivative as a blood marker in a blood sample collected by a primary health examination, which is an in vivo substance present in a living body, and detecting the absence or presence of insulin resistance or IGT for prediabetes on the basis of a result of measurement.

In accordance with the present invention, the insulin resistance or IGT is detected by measuring the methylglyoxal-modified arginine derivative as represented by general formula [I]:

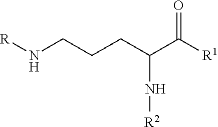

(wherein R is a N-containing heterocyclic group;
R¹ is a hydrogen atom, a hydroxyl group, a protein residue or a peptide residue; and
R² is a hydrogen atom, an acetyl group, a protein residue or a peptide group)
   in a biological sample such as a blood sample.

As the N-containing heterocyclic group as represented by symbol R in the above methylglyoxal-modified arginine derivative [I], there may be mentioned, for example, a pyrimidinyl group as represented by structural formula [VI]:

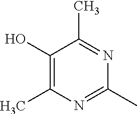

or a hydroimidazolonyl group as represented by structural formula [VII]:

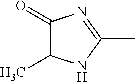

or a hydroimidazolonyl group as represented by structural formula [VIII]:

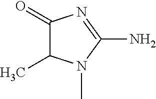

or a hydroimidazolonyl group as represented by structural formula [IX]:

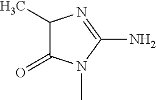

In other words, the methylglyoxal-modified arginine derivative [I] to be used for the present invention may include, for example, an argpyrimidine compound as represented by general formula [II]:

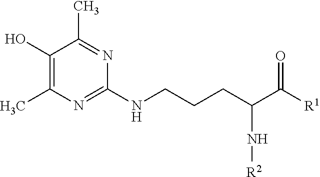

(wherein R¹ and R² have each the same meaning as above),

a hydroimidazolone compound as represented by general formula [III]:

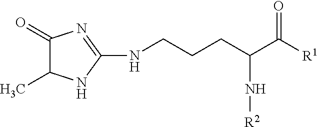

(wherein R¹ and R² have each the same meaning as above),

a hydroimidazolone compound as represented by general formula [IV]:

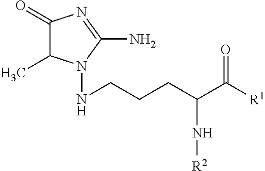

(wherein R¹ and R² have each the same meaning as above),

or a hydroimidazolone compound as represented by general formula [V]:

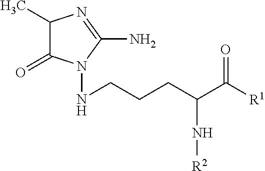

(wherein R¹ and R² have each the same meaning as above).

More specifically, the argpyrimidine compound [II] may be represented by structural formula [VI]:

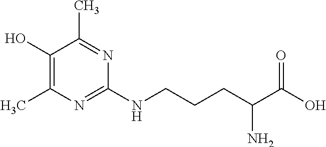 [SMILES: Cc1nc(NCCCC(N)C(=O)O)nc(C)c1O]

and the hydroimidazolone compound [III] may be represented by structural formula [VII]:

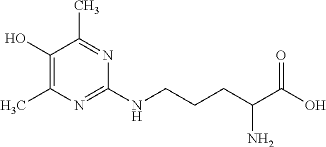 [SMILES: Cc1nc(NCCCC(N)C(=O)O)nc(C)c1O]

In accordance with the present invention, the AP compound may be measured with an assay system using an antibody capable of specifically recognizing the AP compound. As such an antibody, any antibody recognizing specifically the AP compound may be used, whichever is a monoclonal antibody or a polyclonal antibody. As an assay system, there may be preferably used an ELISA system. The use of the ELISA system enables a precise and accurate treatment of the AP compound in a blood sample and a simultaneous treatment of multiple samples. Although any ELISA system conventionally used in the art may be used preferably for the present invention, it is not limited to a particular ELISA system, and there may be used any assay system that uses an antibody capable of specifically recognizing the AP compound.

The ELISA system to be used for the method for the detection of the insulin resistance or IGT for prediabetes according to the present invention may comprise:

a primary antibody reaction step in which the argpyrimidine compound in a sample is reacted with a primary antibody;

an immobilization step in which a blood serum albumin (BSA)-methylglyoxal (MGO) conjugate obtained by a reaction of blood serum albumin (BSA) with the methylglyoxal (MGO) is immobilized;

a MGO-primary antibody reaction step in which the sample treated in the primary antibody reaction step is added to the resulting BSA-MGO conjugate immobilized in the immobilization step to react the MGO of the BSA-MGO conjugate with the primary antibody in the sample; and

a measurement step in which the primary antibody reacted with the MGO in the MGO-primary antibody reaction step is reacted with a labeled secondary antibody, and the labeled secondary antibody is measured on the basis of its coloring.

In accordance with the present invention, the primary antibody is added to the blood sample as a test sample to react the argpyrimidine (AP) compound present in the sample with the primary antibody. As the monoclonal antibody to be used herein as the primary antibody, there may be preferably used the anti-MGO monoclonal antibody disclosed in prior art (for example, see Patent Document Nos. 1, 2 and 3). The antibody to be used for the present invention, however, is not limited to the monoclonal antibody, and there may be used any antibody that can specifically recognize the argpyrimidine structure. A polyclonal antibody may also be used.

The reaction of the methylglyoxal (MGO) with bovine serum albumin (BSA) yields the BSA-MGO conjugate which in turn is immobilized on wells of a plate in a conventional way. To the immobilized BSA-MGO conjugate was added the test sample, thereby reacting the anti-MGO monoclonal antibody present in the test sample with the MGO of the immobilized BSA-MGO conjugate. The anti-MGO antibody reacted with the BSA-MGO conjugate is then reacted with a secondary antibody including, for example, a labeled antibody such as a HRP labeled antibody, and the MGO is then measured on the basis of its coloring.

On the other hand, a solution containing BOC-argpyrimidine is prepared as a standard sample, and it is treated in substantially the same manner as above to cause coloring to measure an amount of argpyrimidine, thereby preparing a standard curve. The amount of argpyrimidine in the test sample may be computed by measuring the amount of argpyrimidine in the test sample on the basis of the standard curve of the standard sample.

In accordance with the present invention, the insulin resistance or IGT for prediabetes may be detected by measuring the argpyrimidine (AP) value using a mouse model or a rat model. As the mouse model, there may be used, for example, a prediabetic mouse formed by aging and a normal mouse as a control mouse. As the rat model, there may be used, for example, a prediabetic rat formed by loading fructose and a normal rat as a control rat.

The value measured by ELISA is generally considered to be variable in accordance with an antibody or a standard substance to be used therefor. And even if the same antibody or standard substance would be measured with ELISA, the resulting values may be varied in accordance with species. Therefore, as the method for the diagnosis of prediabetes or obese diabetes by the measurement of argpyrimidine (AP), there may be used, for example, a method for detecting prediabetes on the basis of a ratio of a normal AP value with respect to a measured AP value of a disease model, in addition to the method based on the measured AP value.

In the case where the prediabetes is detected by detecting the insulin resistance or IGT in the prediabetes on the basis of the argpyrimidine (AP) value measured using the mouse model or the rat model, the case is decided as positive in insulin resistance or IGT when the measured argpyrimidine (AP) value is encompassed within the following values. In addition, the prediabetes is decided on the basis of a range of the AP values.

(1) In the case where the mouse models are used:

When normal mice (6-week-old B6 mice) are used and argpyrimidine (AP) value is in the range of 0.05-0.08 nmol/mg protein or less, insulin resistance or IGT is decided as negative: insulin resistance or IGT (−).

When prediabetic mice (27-week-old B6 mice formed by aging) are used and the argpyrimidine (AP) value is in the range of 0.10-0.14 nmol/mg protein, the insulin resistance or IGT is decided as positive: insulin resistance or IGT (+).

(2) In the case where the rat models are used:

When normal rats are used as a control and the AP value is in the range of 0.10 to 0.13 nmol/mg protein, the insulin resistance or IGT is decided as negative: insulin resistance or IGT (−).

When fructose-loaded (borderline-type) rats are used and the AP value is in the range of 0.13 to 0.30 nmol/mg protein (preferably 0.20), the insulin resistance or IGT is decided as slightly positive: insulin resistance or IGT (+).

The present invention enables a determination as to whether the insulin resistance or IGT is positive or not on the basis of the results of measurement done by the AP measuring method according to the present invention. The present invention can determine the absence or presence of the insulin resistance or IGT from a ratio of the measured AP value with respect to the normal value by setting the normal value to 1. Therefore, the method for the detection of prediabetes according to the present invention can detect prediabetes even if the fasting blood glucose level would be in a normal or substantially normal range.

The present invention enables a monitoring of periodical changes of the insulin resistance or IGT by the above AP measuring method. Further, the present invention can screen substances effective for a prevention or treatment of diabetes, particularly prediabetes, by monitoring a degree of the insulin resistance or IGT by the AP measuring method.

Moreover, the present invention provides an assay kit for the measurement of the insulin resistance or IGT by the above AP measuring method. The assay kit for the measurement of the insulin resistance or IGT according to the present invention may preferably comprise a standard argpyrimidine (AP) compound; an anti-AP antibody, preferably an anti-AP monoclonal antibody, as a primary antibody; an immobilization plate on which the BSA-MGO conjugate consisting of methylglyoxal (MGO) and bovine serum albumin (BSA) is immobilized; an anti-methylglyoxal (MGO) antibody, preferably an anti-MGO monoclonal antibody, as a secondary antibody; a labeled antibody (for example, a HRP-labeled antibody); and a standard curve of a standard sample. The use of the above assay kit for the measurement of the insulin resistance or IGT enables a precise and rapid measurement of the AP compound in a blood sample, leading to a precise and rapid detection of the insulin resistance or IGT in prediabetes. Moreover, the present invention can assess, monitor or screen the insulin resistance or IGT for the detection of prediabetes.

The following is a specific description of the present invention by way of working examples. It is to be understood that the present invention is not interpreted to be limited in any respect by the following working examples, and the following working examples are described hereinafter solely for illustrative purposes and without any intention whatsoever to limit the present invention thereto.

### EXAMPLES

### Example 1

This example describes a process for forming a prediabetic mouse. Five-week-old C57BL/6J mice (hereinafter referred to as “B6 mice”) were purchased from Charles River Laboratories Japan Inc. The mice were housed in a breeding room after receipt and bred under a light and darkness cycle for every 12 hours in the environment in which to eat solid feed (Oriental Yeast Co., Ltd.) and drink tap water any time until they became intended week old. They were then used for experiments.

### Example 2

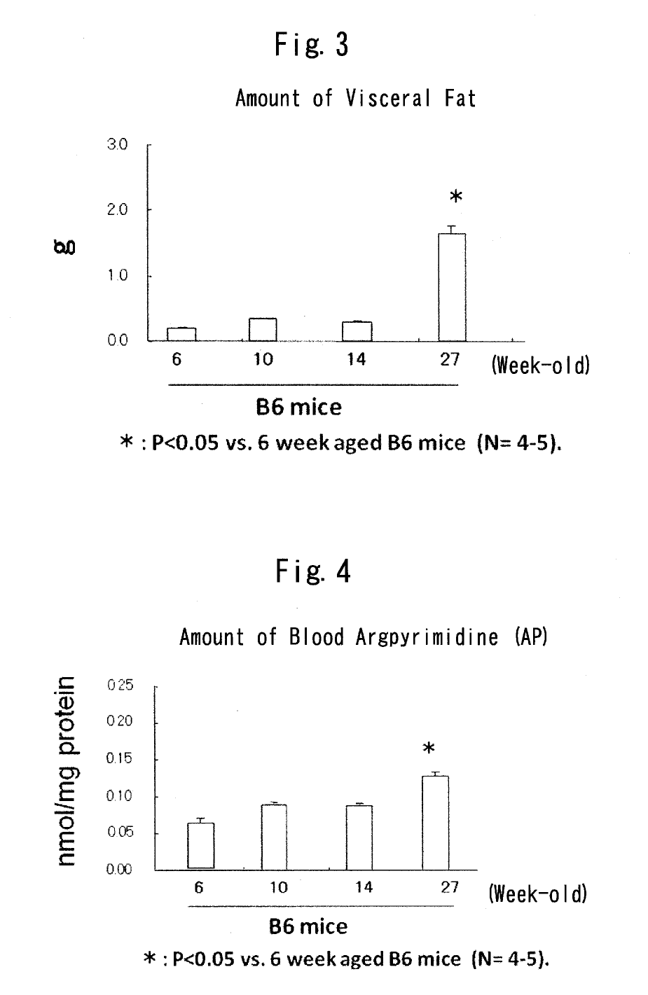

The mouse was anesthetized by intraperitoneally administering a Nembutal injection at a dose of 1 ml per kg of weight, and blood was taken from the heart with a heparin-treated injector after weighing the body weight. The blood collected was centrifuged at 4° C. and 1,000×g for 10 minutes yielding a blood plasma sample. After visceral fat was collected, the mouse was sacrificed. The visceral fat was weighed (see FIG. 3).

### Example 3

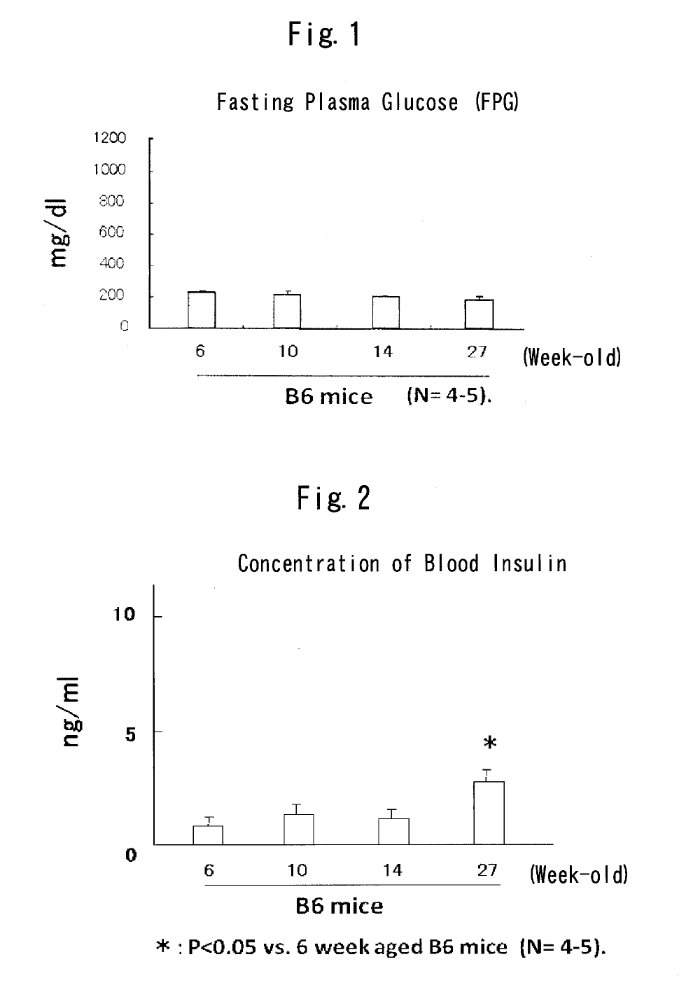

The blood glucose level was measured using the Glutest sensor (Sanwa Kagaku Kenkyusho Co., Ltd.). The blood insulin value was measured using a super-high mouse insulin assay kit (Morinaga Institute of Biological Science, Inc.). The blood glucose and blood insulin values are shown in FIG. 1 and FIG. 2, respectively.

### Example 4

The blood AP was measured in the manner as will be described hereinafter. First, a protein for immobilization was prepared by incubating bovine serum albumin (BSA) (1 μg/ml) and MGO (40 μM) at 37° C. for 24 hours under shading to yield BSA-MGO conjugate. The resulting BSA-MGO conjugate was then diluted with 10 mM phosphate buffer (pH 7.4) and then added to each well of a microplate so as to be contained at a rate of 0.5 μg per well, followed by adding the phosphate buffer to a 96-well microplate at a rate of 100 μl per well and immobilizing the resulting BSA-MGO conjugate to each well by allowing the microplate to stand at 37° C. for 2 hours. The microplate was then washed three times with 0.05% phosphate buffer—Tween (registered trademark) and then blocked with a blocking solution.

Then, the blood plasma sample or the standard solution was reacted with an anti-methylglyoxal (MGO) monoclonal antibody as a primary antibody. The blood plasma sample was diluted with phosphate buffer (pH 7.4) so as to allow the protein to be contained at a rate of 1 μg/50 μl, and 200 μl of the blood plasma sample was reacted with anti-MGO monoclonal antibody diluted 150-fold with 10 mM phosphate buffer (pH 7.4) at 37° C. for 1 hour. The standard solution was prepared so as to contain BOC-argpyrimidine at given concentrations and reacted with the anti-MGO monoclonal antibody diluted 150-fold with 10 mM phosphate buffer (pH 7.4) at 37° C. for 1 hour in substantially the same manner as above.

Thereafter, the resulting reaction mixture of the blood plasma sample or the standard solution with the anti-MGO monoclonal antibody was subjected to a competition reaction with the immobilized protein by adding the mixture to each well of the plate at a rate of 100 μl and allowing the microplate to stand at 37° C. for 1 hour. After the reaction was completed, the microplate was washed with a 0.05% phosphate buffer—Tween (registered trademark) mixture, and 100 μl of biotin-labeled secondary antibody diluted 10,000-fold with a blocking solution was added to each well and allowed to stand at 37° C. for 1 hour. After washing, 100 μl of a coloring solution was added to each well of the microplate and then allowed to stand at room temperature for 15 minutes, followed by adding 100 μl of 0.5 M sulfuric acid to each well to suspend the reaction. Within 15 minutes after the suspension of the reaction, absorbance was measured at 450 nm. The result of measurement is shown in FIG. 4.

From the result indicated in FIG. 1 about an advancement of prediabetes for the B6 mice by aging, it is noted that there is no difference in the fast blood glucose level between the 10-27-week-old B6 mice and the 6-week-old mice. As shown in FIG. 2, however, the blood insulin concentration of the 27-week-old B6 mice was significantly increased by more than three times than that of the 6-week-old B6 mice. Further, as shown in FIG. 3, the amount of the visceral fat of the 27-week-old B6 mice was also increased remarkably compared to that of the 6-week-old B6 mice. Moreover, as shown in FIG. 4, the blood AP value of the 27-week-old B6 mice was significantly increased at maximum roughly by two times compared to that of the 6-week-old B6 mice.

As described above, it was observed that, although the blood glucose level of the 27-week-old B6 mice is not varied compared to the 6-week-old B6 mice, the insulin concentration of the 27-week-old B6 mice was increased to a remarkable extent and the visceral fat was accumulated which is considered as a major cause of the insulin resistance. Therefore, the 27-week-old B6 mice developed an apparent prediabetes by aging, compared to the 6-week-old B6 mice, and an increase in the blood AP recognized in the 27-week-old B6 mice may be considered to be useful for the detection of the insulin resistance or IGT, thereby utilizing it as a parameter for a diagnosis of prediabetes.

### Example 5

After a group of 6-week-old male SD rats was bred for 1 week under a light and dark cycle for 12 hours each in the environment in which they could eat solid feed (Oriental Yeast Co., Ltd.) and drink tap water any time, it was used for experiment. Separately, a group of model IR rats was prepared by allowing a free drinking of 15% fructose aqueous solution instead of tap water for 4 weeks (hereinafter referred to as “fructose group”). On the other hand, another group of rats was prepared by allowing a free drinking of tap water (hereinafter referred to as “control group”).

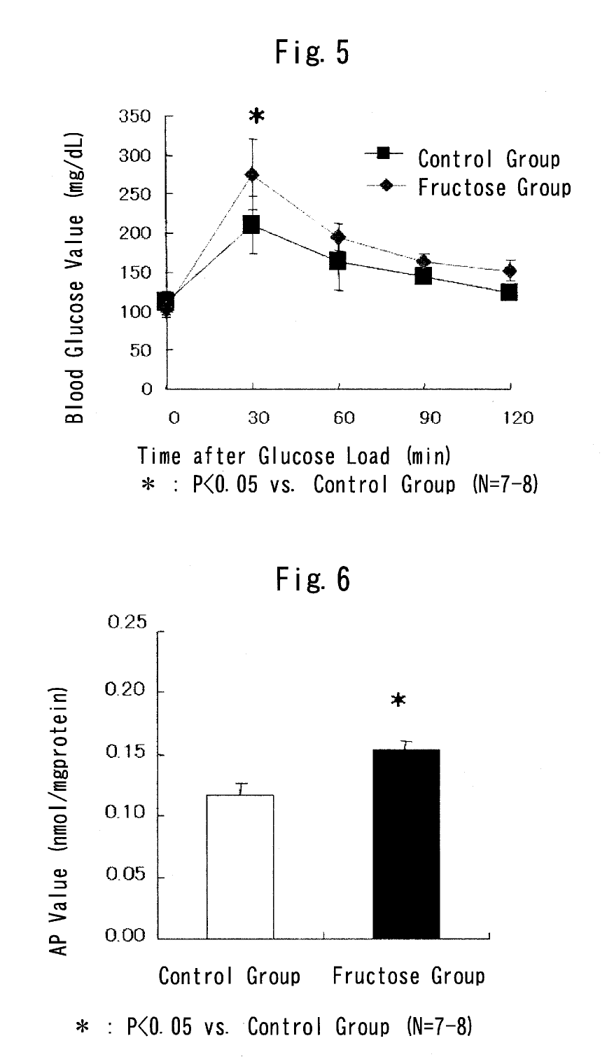

Experiments were carried out using the above rats to investigate the glucose tolerance by loading glucose. The groups of the rats which were bred for 4 weeks by allowing a free drinking of 15% fructose solution and tap water, respectively, were fasted for 19 hours, and blood samples were collected from their caudal veins to measure AP and fasting blood glucose level at minute 0. Then, glucose (2 g/kg) was intraperitoneally administered to each rat, followed by collecting a blood sample from the caudal vein in 30 to 120 minutes after administration and measuring the blood glucose level for each sample using Glutest Sensor (Sanwa Kagaku Kenkyusho Co., Ltd.). The results for periodical variations in the blood glucose levels between the fructose group and the control group are shown in FIG. 5 in which the blood glucose levels are indicated on the vertical axis and a time elapse after the glucose load is indicated on the horizontal axis. FIG. 5 shows a significant rise of the blood glucose level in 30 minutes after the glucose load in the fructose group compared to the control group, thereby recognizing IGT, although there is no change in the fasting blood glucose level between the fructose group and the control group. This indicates that the fructose group caused prediabetes.

### Example 6

An argpyrimidine (AP) amount was measured from a plasma sample of the rat collected and treated separately in Example 5. The plasma sample gathered from the caudal vein of the rat was prepared with a phosphate buffer (pH7.4) so as to contain the proteins in the amount of 1 μg/50 μl and then treated in substantially the same manner as in Example 4, thereby measuring the plasma AP amount by reading absorbance at 420 nm. FIG. 6 shows the result indicating a significant increase in the plasma AP amount for the fructose group compared to the control group.

From the above experiment results, an appearance of prediabetes was confirmed in the fructose group and an increase in the blood AP amount was also confirmed therein. Although it has been already known that mice may cause hyperinsulinemia by administration of fructose demonstrating insulin resistance and IGT, the experiment results shown by the present invention indicate that the measurement for the blood AP value according to the present invention may be useful for the detection of the insulin resistance or IGT and it can be applied to a diagnosis of prediabetes.

### INDUSTRIAL APPLICABILITY

The method for the measurement of AP according to the present invention enables a measurement of AP by using a sample for measuring blood glucose to be used for the primary health examination and a simultaneous treatment of multiple samples in a precise manner. If the examination of prediabetes could be performed by measuring AP in the primary health examination, an early detection of prediabetes can be achieved which is otherwise difficult to be detected solely by measuring AP. Further, if the diagnosis of prediabetes could be achieved by the primary health examination, it is made possible to do without a binding time for those who receive examination and avoid a risk that could be caused by loading glucose by OGTT in the secondary health examination. This may serve in an early treatment of prediabetes and a prevention of prediabetes from a development into diabetes or complications. As a result, the present invention is expected to save an immeasurable amount of expenses for treatment of diabetes and complications

1. A method for the detection of prediabetes comprising a detection of prediabetes on the basis of a result of an examination of a presence or absence of insulin resistance or impaired glucose tolerance (IGT) in prediabetes by measuring a methylglyoxal-modified arginine derivative as represented by general formula [I]: 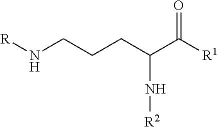
(wherein R is an N-containing heterocyclic group;
R1 is a hydrogen atom, a hydroxyl group, a protein residue or a peptide residue;
R2 is a hydrogen atom, an acetyl group, a protein residue or a peptide residue).

2. The method for the detection of prediabetes as claimed in claim 1, wherein said N-containing heterocyclic group as represented by symbol R is a pyrimidinyl group as represented by structural formula [VI]: 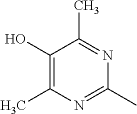
or a hydroimidazolonyl group as represented by structural formula [VII]: 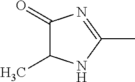
or a hydroimidazolonyl group as represented by structural formula [VIII]: 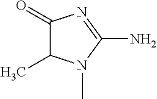
or a hydroimidazolonyl group as represented by structural formula [IV]:

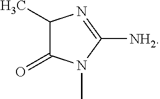

3. The method for the detection of prediabetes as claimed in claim 1, wherein said methylglyoxal-modified arginine derivative [I] is an argpyrimidine compound as represented by general formula [II]: 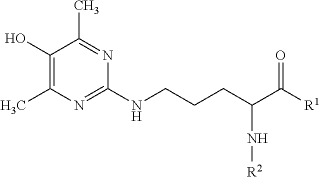
(wherein R¹ and R² have each the same meaning as above),
or a hydroimidazolone compound as represented by general formula [III]: 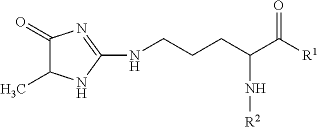
(wherein R¹ and R² have each the same meaning as above),
or a hydroimidazolone compound as represented by general formula [IV]: 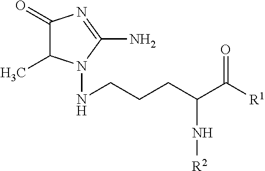
(wherein R¹ and R² have each the same meaning as above),
or a hydroimidazolone compound as represented by general formula [V]: 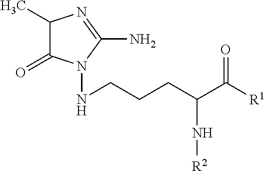
(wherein R¹ and R² have each the same meaning as above).

4. The method for the detection of prediabetes as claimed in claim 1, wherein said methylglyoxal-modified arginine derivative [I] is argpymidine as represented by structural formula [VI]: 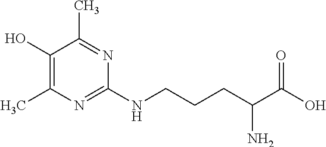 [SMILES: Cc1nc(NCCCC(N)C(=O)O)nc(C)c1O]
or a hydroirnidazolone as represented by structural formula [VII]:

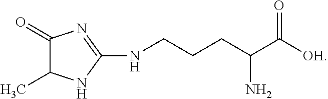 [SMILES: CC1NC(NCCCC(N)C(=O)O)=NC1=O]

5. The method for the detection of prediabetes as claimed in claim 1, wherein said methylglyoxal-modified arginine derivative is measured by an assay system using an antibody capable of specifically recognizing the methylglyoxal-modified arginine derivative.

6. The method for the detection of prediabetes as claimed in claim 5, wherein said antibody is a monoclonal antibody or a polyclonal antibody recognizing specifically the methylglyoxal-modified arginine derivative.

7. The method for the detection of prediabetes as claimed in claim 5, wherein said assay system is an ELISA system.

8. The method for the detection of prediabetes as claimed in claim 1, which comprises:
a primary antibody reaction step for reacting a methylglyoxal-modified arginine derivative in a sample with a primary antibody;
an immobilization step for immobilizing a serum albumin—methylglyoxal conjugate obtained by a reaction of serum albumin with a methylglyoxal;
a methylglyoxal—primary antibody reaction step for reacting the methylglyoxal of the serum albumin—methylglyoxal conjugate immobilized in the immobilization step with the primary antibody in the sample; and
a measurement step for measuring a labeled secondary antibody by reacting the primary antibody reacted in the methylglyoxal—primary antibody reaction step with the secondary antibody.

9. (canceled)

10. The method for the detection of prediabetes as claimed in claim 1, wherein said method qualifies an amount of methylglyoxal measured in the sample on the basis of a calibration curve of a standard sample.

11. The method for the detection of prediabetes as claimed in claim 1, comprising measuring the methylglyoxal-modified arginine derivative in a blood sample.

12. The method for the detection of prediabetes as claimed in claim 1, wherein said standard blood glucose level of the blood sample is less than 110 mg/dl or less than 126 mg/dl.

13. An assay kit for the measurement for prediabetes comprising an assay kit for measuring insulin resistance and IGT in prediabetes as claimed in claim 1 by measuring a methylglyoxal-modified arginine derivative and detecting the insulin resistance and IGT in prediabetes on the basis of a result of measurement of the methylglyoxal-modified arginine derivative, which comprises:
a methylglyoxal-modified arginine derivative; a primary antibody; an immobilized plate on which a methylglyoxal—bovine serum albumin (BSA) is immobilized; a secondary antibody; a labeled antibody (for example, a HRP labeled antibody, etc.); and a standard curve of a standard sample.
